(19) United States
(12) Patent Application Publication
(10) Pub. No.: US 2007/0270756 A1
(43) Pub. Date: Nov. 22, 2007

(54) Needleless access port valves

(76) Inventors: Peter Peppel, Nazareth, PA (US); Kristopher Kundra, Lambertville, NJ (US)

Correspondence Address: CHRISTIE, PARKER & HALE, LLP, PO BOX 7068, PASADENA, CA, 91109-7068, US

(21) Appl. No.: 11/438,809

(22) Filed: May 22, 2006

(51) Int. Cl. A61M 5/178

(52) U.S. Cl. 604/167.06

(57) ABSTRACT

Needleless access port valves are generally discussed herein with particular discussions extended to needleless access port valves comprising a piston comprising an integrated gilled sheath. In accordance with aspects of the present invention, the sheath is secured to a valve housing and provides the needed recoil function to return a core from a second position to a first position to close the valve upon removal of a medical implement.

Needleless access port valves are generally discussed herein with particular discussions extended to needleless access port valves comprising a piston comprising an integrated gilled sheath.

### BACKGROUND

Needleless access port valves are widely used in the medical industry for accessing an IV line and/or the internals of a patient or subject. Generally speaking, prior art valves utilize a housing in combination with a moveable internal plug or piston to control the flow of fluid through a valve. The plug or piston may be moved by a syringe or a medical implement to open the inlet of the valve for accessing the interior cavity of the valve. When a fluid is delivered through the valve, fluid flow typically flows around the outside of the plug or piston in the direction towards the outlet. Upon removal of the syringe or medical implement, the plug or piston returns to its original position, either un-aided or aided by a biasing means, such as a spring or a diaphragm.

In some prior art valves, when the syringe or medical implement pushes the plug or piston, the plug or piston is pierced by an internal piercing device, such as a spike. The spike typically incorporates one or more fluid channels for fluid flow flowing through the pierced piston and then through the fluid channels in the spike. In yet other prior art valves, a self-flushing or positive flush feature is incorporated to push residual fluids confined inside the interior cavity of the valve to flow out the outlet when the syringe or medical implement is removed.

While prior art needleless access port valves are viable options for their intended applications, there remains a need for alternative needleless access port valves.

### SUMMARY

The present invention may be implemented by providing a needleless injection port valve comprising a valve housing defining an interior cavity having an inlet and an outlet, a piston comprising a core and a sheath positioned in the interior cavity of the housing by securing a perimeter section of the sheath in a bore proximate two mating surfaces on the valve housing; and wherein the sheath comprises a plurality of seals each comprising resilient surface to resilient surface contact.

In accordance with other aspects of the present invention, there is provided a needleless injection port valve comprising a valve housing defining an interior cavity and a piston comprising a core and a sheath surrounding, at least in part, the core positioned in the interior cavity of the housing; the housing comprises a inlet section comprising an inlet opening, an outlet section comprising an outlet opening, and a body section attached to the inlet section and the outlet section; the body section having a body upper section, a body center section, and a body lower section all having a respective cross-sectional dimension, and wherein the cross-sectional dimension of the body center section is less than the cross-sectional dimensions of the body upper section and body lower section along a cross-sectional side view of the valve.

In yet other aspects of the present invention, there is provided a needleless injection port valve comprising a valve housing defining an interior cavity and a piston comprising a core and a sheath attached to a lower section of the core and extending proximally along at least a portion of the core; the piston being positioned in the interior cavity by wedging a perimeter section of the sheath in between a shoulder defined by an inlet nozzle section and a shoulder defined by an upper housing chamber; wherein the piston comprises at least one gill located on the sheath; said gill having a first configuration corresponding to a first valve position in which a first resilient surface contacts a second resilient surface and having a second configuration corresponding to a second valve position in which the first resilient surface is spaced apart from the second resilient surface.

In yet another aspect of the present invention, there is provided a plurality of ribs connected to a core and a sheath for increasing the returning force in returning the core from a used position to a ready position.

In still yet another aspect of the present invention, there is provided separate threaded collar mechanically coupled to an outlet for providing a threaded male Luer connector.

In a further aspect of the present invention, there is provided a body section over-molded to an upper valve body chamber and a lower valve body chamber defining a valve cavity.

Other aspects and variations of the valve assemblies summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is a semi-schematic perspective view of an injection port valve provided in accordance with aspects of the present invention;

FIG. 2 is a semi-schematic top view of the valve of FIG. 1;

FIG. 3 is a semi-schematic cross-sectional side view of the valve of FIG. 2 taken along line A-A;

FIG. 4 is a semi-schematic cross-sectional side view of the valve of FIG. 3 taken along line B-B;

FIG. 5 is a semi-schematic perspective view of a piston provided in accordance with aspects of the present invention;

FIG. 6 is a semi-schematic perspective view of an injection port valve provided in accordance with aspects of the present invention in a used position;

FIG. 7 is a semi-schematic top view of the valve of FIG. 6;

FIG. 8 is a semi-schematic cross-sectional side view of the valve of FIG. 7 taken along line C-C;

FIG. 9 is a semi-schematic cross-sectional side view of the valve of FIG. 8 taken along line D-D; and

FIG. 10 is a semi-schematic perspective view of the piston of FIG. 5 in a used configuration.

### DETAILED DESCRIPTION

The detailed description set forth below in connection with the appended drawings is intended as a description of the presently preferred embodiments of needleless access port valves or backcheck valves (herein “valves”) provided in accordance with aspects of the present invention and is not intended to represent the only forms in which the present invention may be constructed or utilized. The description sets forth the features and the steps for constructing and using the valves of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

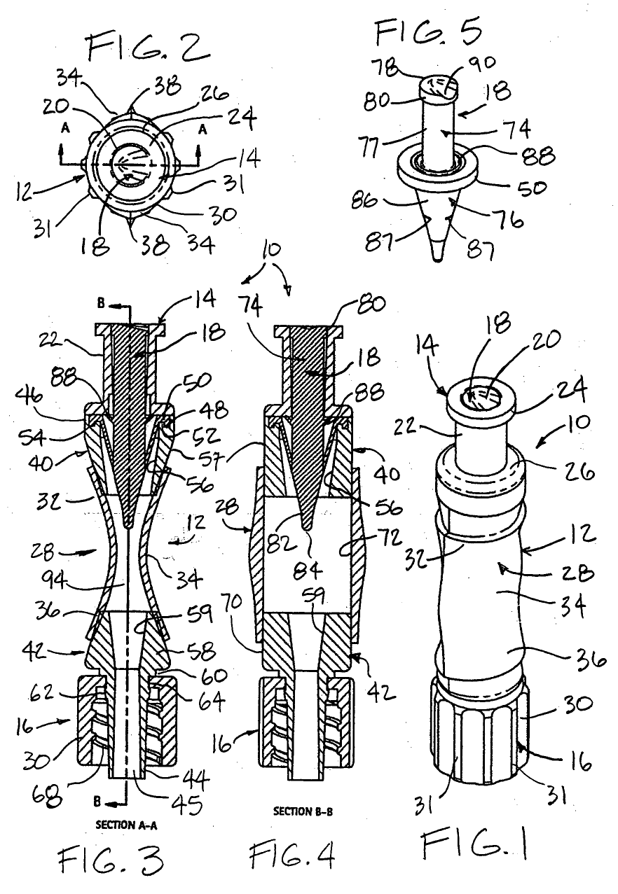

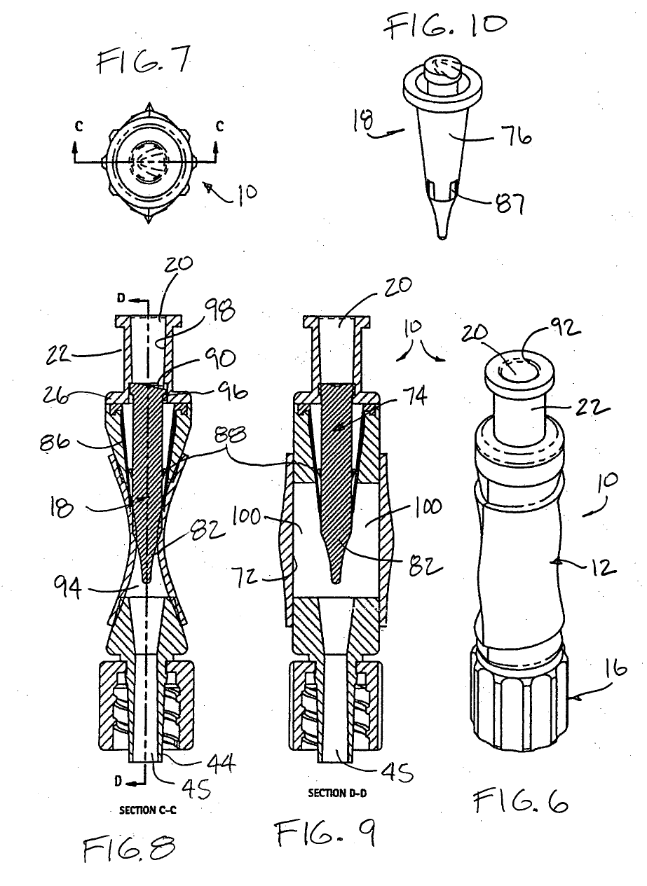

Turning now to FIG. 1, a semi-schematic perspective view of a valve provided in accordance with aspects of the present invention is shown, which is generally designated 10. In one exemplary embodiment, the valve 10 comprises a valve housing 12 comprising an inlet 14 and an outlet 16. The inlet 14 is adapted to receive a first medical implement (not shown), such as a syringe, an IV tubing adapter, and the like, for delivering fluid from the first medical implement through the valve 10 and out the outlet 14, which is adapted to connect to a second medical implement, such as a tubing or a catheter assembly, for delivering the same to a patient or subject. In one exemplary embodiment, the inlet and the outlet both incorporate a standard luer taper.

The valve 10 is shown in a first or closed position (FIG. 1) having a piston 18 blocking the opening 20 defined by an inlet nozzle 22. As further discussed below, when the piston 18 is in the first position, a seal located on the piston 18 seals against an interior surface of the inlet nozzle 22 to close fluid communication between the inlet 14 and the outlet 16. In one exemplary embodiment, the inlet 14 comprises a female luer slip and an inlet lip 24 having a generally ring shaped structure. Alternatively, the inlet 14 may incorporate luer threads without deviating from the spirit and scope of the present invention.

Also shown in FIG. 1 is an inlet shoulder 26, a valve body 28, and a threaded collar 30 comprising a plurality of ridges 31, which surrounds the outlet nozzle 44 (FIG. 3). As further discussed below, the valve body 28 comprises an upper valve body section 32, a middle valve body section 34, and a lower valve body section 36 and wherein the middle valve body section 34 comprises a cross-sectional dimension that is less than the cross-sectional dimensions of the upper and the lower valve body sections 32, 36. In one exemplary embodiment, the valve body 28 is integrally formed with two creases 38 defining two apexes (FIG. 2), which resembles a partially crushed cylindrical tube having different cross-sectional dimensions along the length of the tube (See, e.g., FIG. 3). In one exemplary embodiment, the valve body 28 is made from a rigid or semi-rigid plastic of a semi-crystalline polymer type, such as polycarbonate, polypropylene, polyethylene, and nylon and more preferably from an elastomeric plastic of a thermoplastic elastomer (TPE) type such as the copolyamide (COPA) family of thermoplastic elastomers. In a preferred embodiment, the COPA is copolyamide thermoplastic elastomer having a commercial trade name PEBAX®. However, other TPEs may also be used to make the valve body 28, including thermoplastic polyurethanes (TPUs), styrenic thermoplastic elastomers, thermoplastic polyolefins (TPOs), copolyesters (COPEs), and thermoplastic vulcanizate elastomeric alloys (TPVs). Optionally, the TPEs may be cross-linked either chemically or by irradiation to alter their characteristics.

FIG. 2 is a top view of the valve 10 of FIG. 1. The piston 18 is shown occupying the opening 20 of the inlet nozzle 22 and occluding the opening from fluid flow when in the first position. The two creases 38 and part of the middle body section 34 are also shown, projecting beyond the periphery of the outlet collar 30.

FIG. 3 is a cross-sectional side view of the valve of FIG. 2 taken along line A-A. In one exemplary embodiment, the valve housing 12 comprises an upper housing chamber 40 coupled to a body section 28 and to a lower housing chamber 42, which comprises an outlet nozzle 44 comprising an outlet opening 45. In a preferred embodiment, the body section 28 is over-molded to the upper and lower housing chambers 40, 42, which are preferably made from a hard thermoplastic, such as, for example, polycarbonate, ABS, or acrylic. Alternatively, the parts may be glued together rather than over-molded. The upper housing chamber 40 comprises a perimeter rim 46 and a mating seat 48 for matingly engaging a piston flange 50 on the plunger 18. In one exemplary embodiment, the mating seat 48 comprises an inner rim 52 and together with the perimeter rim 46 define a groove 54. The piston flange 50 is positioned in the groove 54 and is secured thereto by fixing the inlet shoulder 26 to the perimeter rim 46. In one embodiment, the shoulder 26 and the rim 46 are welded, using a laser or high frequency welding, to maintain a more permanent connection. Alternatively, glue or adhesive may be used to bond the shoulder 26 and the rim 46 together to retain the piston flange therein.

The upper housing chamber 40 comprises an interior surface defining a bore 56 that tapers slightly inwardly in the distal direction and an exterior wall 57 that also tapers inwardly in the distal direction. In a preferred embodiment, the slope of the exterior wall 57 is greater than the slope of the interior wall 56 so that the upper housing chamber 40 has a greater wall thickness near its proximal end than at its distal end.

The body section 28, in the cross-sectional side view of FIG. 3, comprises an hourglass configuration with the upper valve body section 32 and the lower valve body section 36 being larger than the middle valve body section 34. Exteriorly, the lower valve body section 36 tapers outwardly as it extends distally to mate with a similarly outwardly tapered hub section 58 of the lower housing chamber 42. Interiorly, the wall surface of the tapered hub section 58 tapers inwardly as it extends distally to communicate with a generally cylindrical lumen of the outlet nozzle 44.

Exteriorly, the lower housing chamber 42 comprises an upper shoulder section 60 and a lower shoulder section 62 defining a groove 64 therebetween. The groove 64 functions as a female detent for mating engaging with a male detent or proximal cylindrical opening 66 of the threaded collar 30. A tapered ramp just distal of the lower shoulder section 62 may be incorporated to facilitate insertion of the outlet nozzle 44 into the proximal opening 66 of the collar 30 and for the opening to slide over and engage the female detent 64. The collar 30, having interior threads 68, may be made from a hard plastic material, such as, for example, polycarbonate or ABS.

FIG. 4 is a cross-sectional side view of the valve of FIG. 3 taken along line B-B. The upper and lower housing chambers 40, 42 are shown with exterior wall surfaces 57, 70 that are generally constant as measured from a center axis defined along a lengthwise direction of the valve 10. However, the interior wall surfaces 56, 59 of the upper and lower housing chambers 40, 42 maintain similar taper as that shown in the cross-sectional side view of FIG. 3, i.e., they are symmetrical. The interior wall surface 72 of the body section 28 is also generally constant as measured from the center axis defined by the lengthwise direction of the valve.

In one exemplary embodiment, the piston 18 comprises an elongated core 74 and a sheath 76 comprising a piston flange 50. In a preferred embodiment, the elongated core 74 and the sheath 76 are integrally formed from a medical grade silicone material. However, other rubber materials may be used without deviating from the spirit and scope of the present invention, including polyisoprene. In one exemplary embodiment, the piston 18 incorporates a self-lubricating material for facilitating movement of the core 74 from a first position to a second position and vice versa. The self-lubricating material reduces friction between the interface of the core 74 and sheath 86 and the interior surface of the inlet nozzle 22. In one exemplary embodiment, the self-lubricating material is a two-part self-lube liquid silicone rubber. The two-part self-lube silicone rubber is commercially available from Nusil Silicone Technology of Santa Barbara, Calif. Various aspects of the self-lube liquid silicone rubber are described in U.S. Pat. No. 6,871,838, filed Apr. 3, 2003, the contents of which are expressly incorporated herein by reference as if set forth in full.

The core 74 comprises a body section 77 comprising an outer diameter and a head section 78 comprising an outer perimeter rim 80 having an outer diameter larger than the diameter of the body section 77. In a preferred embodiment, the diameter of the outer perimeter rim 80 is also larger than the inner diameter of the inlet nozzle 22 for sealing against the interior surface of the inlet nozzle to seal the valve 10 when the piston is in the closed position. In one exemplary embodiment, a 0.5 mil to about a 2.5-mil total interference fit is incorporated between the outer perimeter rim 80 and the interior diameter of the inlet nozzle 22. The core 74 comprises a tapered lower section 82 terminating in a rounded distal end point 84 (FIG. 4). The tapered lower section 82 provides clearance between the core 74 and the interior surface 56 of the upper housing chamber 40 so that the plunger may move 18 between a first position to a second position and vice-versa with little or no obstruction.

With reference to FIG. 5 in addition to FIGS. 3 and 4, the sheath 76 comprises an outer shroud 86 and a plurality of inner ribbing materials 88, which are positioned adjacent the piston flange 50 and are connected to the flange and to the core 74. In one exemplary embodiment, four equally spaced apart inner ribbing materials 88 are incorporated with each connected to both the core 74 and the piston flange 50 and having a gap or a flow path therebetween. The sheath 76 and the ribbing materials 88, both made from a resilient material, provide the necessary returning forces to return the core 74 to its closed position from an open position, as further discussed below. However, the sheath alone 76 may provide the necessary resilient biasing force without the ribbing materials 88 by varying the resiliency or thickness of the sheath.

A trough 90 is incorporated on the surface of the head section 78. The trough resembles a trench or an indentation and is configured as a flow path for fluid flow flowing from a syringe or a medical implement (not shown) through the valve housing 12, or vice versa. In an alternative embodiment, a plurality of protrusions are incorporated instead of or in addition to the trough 90 to provide the necessary flow paths on the top surface of the head section 78.

In one exemplary embodiment, two or more gills 87 are incorporated on the sheath 76, with four equally spaced apart gills being more preferred. The gills 87 are formed by making small generally horizontal incisions on the sheath 87, horizontal as compared to the axis defined by the core 74. In one exemplary embodiment, the gills are cut after a the piston has been molded. In another exemplary embodiment, the cut gills undergo a post mold mechanical setting to set the slit. The gills 87 are in a closed position when the piston 18, and hence the valve 10, is in a first or closed position. In the closed position, no fluid will flow from a location in between the core 74 and the sheath 76 to a position external to the sheath 76, and vice-versa.

FIG. 6 is a semi-schematic perspective view of the valve 10 provided in accordance with aspects of the present invention in a second or used position. In the used position, the piston 18 is urged inwardly into the valve housing 12 by a medical implement (not shown), such as a syringe, to open fluid communication between the inlet opening 20 and the outlet opening 45. A chamfer interior edge 92 is incorporated for facilitating insertion of the medical implement in the event of a misalignment between the tip of the medical implement and the opening 20.

FIG. 7 is a top view of the valve 10 of FIG. 6 in the used position.

FIG. 8 is a cross-sectional side view of the valve of FIG. 7 taken along line C-C. The piston 18 is shown urged inwardly into the interior cavity 94 of the valve housing 12 by a medical implement (not shown). The piston 18 is moved a sufficient amount by the medical implement so that the trough 90 coincide with a plurality of internal flow channels 96. Fluid expelled from a medical implement is configured to flow over the trough 90, then in between the plurality of flow channels 96 and then onwards through the outlet nozzle 44. In one exemplary embodiment, the plurality of internal flow channels 96 are formed by incorporating indentations on the interior surface 98 of the inlet nozzle 22. Preferably, three or more equally spaced apart indentations are incorporated on the interior surface 98 adjacent the shoulder or flange 26 with four indentations being more preferred.

The distal movement of the piston 18 towards the outlet nozzle 44 by the medical implement stretches the sheath 86 and the inner ribbing materials 88. With reference to FIG. 10, which depicts the piston 18 in a second or used position outside of the valve body 12, the stretched sheath 86 causes the gills 87 to expand. Thus, when the plunger 18 is in the second position, fluid communication is opened between the inlet opening 20 and the outlet opening 45 through the gills 87. Hence, it accordance with aspects of the present invention, there is provided a plurality of spaced apart seals 87 comprising resilient surface to resilient surface contact for sealing the valve 10 and terminating fluid communication between the inlet opening 20 and the outlet opening 45. In the piston 18 first position (FIGS. 3 & 4), fluid communication between the inlet opening 20 and the outlet opening 45 is further prevented by the surface contact between the outer perimeter rim 80 on the piston core 74 compressing against the interior surface of the inlet nozzle 22.

FIG. 9 is a semi-schematic cross-sectional side view of the valve 10 of FIG. 8 taken along line D-D. In the view shown, the tapered lower end 82 of the core 74 is spaced apart from the interior surface 72 by gaps 100. The gaps 100 provide flow space for fluid flow from between the inlet opening 20 and the outlet opening 45.

The valve 10 or piston 18 is moved from the second used position to its first position by simply removing the force exerted on the piston. The material elasticity of the sheath 86 and of the internal ribbing materials 88 (FIGS. 4, 8, and 9) recoil as the force is removed to return to its less stretched state. Thus, the valve moves from the FIGS. 8/9 position to the FIGS. 3/4 position by simply removing the medical implement from the inlet 14. In one exemplary embodiment, the valve is essentially a neutral valve in that no noticeable net fluid flow into or out of the valve may be noticeable upon moving the piston from the second used position to its first position. However, the valve may be made a positive flush valve (i.e., a small amount of fluid is expelled out of the outlet upon moving the piston from the second position towards the first position) by ensuring a decrease in fluid volume space inside the valve when the piston moves from the second position towards the first position.

Although limited embodiments of the needleless access valve assemblies and their components have been specifically described and illustrated herein, many modifications and variations will be apparent to those skilled in the art. For example, the inlet may incorporate a luer lock, the outlet may simply be a luer slip, the housing material could be opaque or semi-opaque, the various dimensions can vary, exterior angles and curvatures incorporated for aesthetic appeal, etc. Accordingly, it is to be understood that the valve assemblies and their components constructed according to principles of this invention may be embodied other than as specifically described herein. The invention is also defined in the following claims.

What is claimed is:

1. A needleless injection port valve comprising a valve housing defining an interior cavity having an inlet and an outlet, a piston comprising a core and a sheath positioned in the interior cavity of the housing by securing a perimeter section of the sheath in a bore proximate two mating surfaces on the valve housing; and wherein the sheath comprises a plurality of seals each comprising resilient surface to resilient surface contact.

2. The needleless injection port valve of claim 1, wherein the inlet comprises an inlet nozzle comprising a flange attached to an upper chamber comprising a distally directed taper.

3. The needleless injection port valve of claim 2, where the bore is bounded by the flange and the upper chamber.

4. The needleless injection port valve of claim 1, wherein the plurality of seals comprise each comprise a slit.

5. The needleless injection port valve of claim 1, wherein the valve housing comprises a valve body comprising an hourglass configuration.

6. The needleless injection port valve of claim 1, further comprising a rib attached to both the core and the sheath.

7. The needleless injection port valve of claim 1, wherein the core comprises an upper surface comprising trough.

8. The needleless injection port valve of claim 1, wherein the inlet comprises a Luer taper.

9. The needleless injection port valve of claim 1, wherein the piston is made from at least one of a silicone and a polyisoprene material.

10. The needleless injection port valve of claim 1, wherein the piston is made from a self-lubricating material.

11. A needleless injection port valve comprising a valve housing defining an interior cavity and a piston comprising a core and a sheath surrounding, at least in part, the core positioned in the interior cavity of the housing; the housing comprises a inlet section comprising an inlet opening, an outlet section comprising an outlet opening, and a body section attached to the inlet section and the outlet section; the body section having a body upper section, a body center section, and a body lower section all having a respective cross-sectional dimension, and wherein the cross-sectional dimension of the body center section is less than the cross-sectional dimensions of the body upper section and body lower section along a cross-sectional side view of the valve.

12. The needleless injection port valve of claim 11, wherein the sheath comprises a perimeter section, and wherein the perimeter section is compressed between two mating surfaces on the inlet section.

13. The needleless injection port valve of claim 11, wherein the body section is made from a thermoplastic elastomer material.

14. The needleless injection port valve of claim 11, further comprising a plurality of slits positioned on the sheath.

15. The needleless injection port valve of claim 11, further comprising a plurality of ribs connected to both the core and the sheath.

16. The needleless injection port valve of claim 11, wherein the inlet section defines a bore and wherein the sheath is attached to the bore.

17. A needleless injection port valve comprising a valve housing defining an interior cavity and a piston comprising a core and a sheath attached to a lower section of the core and extending proximally along at least a portion of the core; the piston being positioned in the interior cavity by wedging a perimeter section of the sheath in between a shoulder defined by an inlet nozzle section and a shoulder defined by an upper housing chamber; wherein the piston comprises at least one gill located on the sheath; said gill having a first configuration corresponding to a first valve position in which a first resilient surface contacts a second resilient surface and having a second configuration corresponding to a second valve position in which the first resilient surface is spaced apart from the second resilient surface.

18. The needleless injection port valve of claim 17, wherein the piston is made from at least one of a silicone and a polyisoprene material.

19. The needleless injection port valve of claim 17, wherein the valve housing comprises a body section having a hourglass configuration.

20. The needleless injection port valve of claim 17, wherein the at least one gill provides a fluid flow channel when the piston is in the second valve position.
